(19) United States
(12) Patent Application Publication
(10) Pub. No.: US 2026/0248512 A1
(43) Pub. Date: Aug. 27, 2026

(54) ANVIL CONNECTION DETECTION OF CIRCULAR STAPLER

(71) Applicant: Cilag GmbH International, Cincinnati, OH (US)

(72) Inventors: Matthew HILL, Cincinnati, OH (US); Andrew BECKMAN, Cincinnati, OH (US); Ryan CLAY, Cincinnati, OH (US); Christopher DENZINGER, Cincinnati, OH (US); Evan CARRICO, Cincinnati, OH (US); Cole IVEY, Cincinnati, OH (US); Angela EISEL, Cincinnati, OH (US); Maximilian REESE, Cincinnati, OH (US); Kris KALLENBERGER, Cincinnati, OH (US); Mark OVERMYER, Cincinnati, OH (US)

(73) Assignee: Cilag GmbH International, Cincinnati, OH (US)

(21) Appl. No.: 19/545,339

(22) Filed: Feb. 20, 2026

Related U.S. Application Data
(60) Provisional application No. 63/761,653, filed on Feb. 21, 2025.

(51) Int. Cl. A61B 17/115; A61B 17/00; A61B 90/00

(52) U.S. Cl. CPC A61B 17/1155; A61B 90/06; A61B 2017/00075; A61B 2090/066

(57) ABSTRACT

A circular stapler for cutting and applying one or more surgical staples to tissue is disclosed. The circular stapler includes various forms of feedback systems that are configured to provide feedback to the surgeon that an anvil of the circular stapler is properly attached to a trocar of the circular stapler before closing and firing of the circular stapler. The various forms of feedback systems can include one or more of hard stops, friction detection, torque detection, acceleration detection, signal on/off detection, and audible detection, among other options for detecting that the anvil is properly attached to the trocar of the circular stapler before closing and firing of the circular stapler.

### CROSS REFERENCE TO RELATED APPLICATIONS

This application claims the benefit of U.S. Provisional Application No. 63/761,653, filed Feb. 21, 2025, which is incorporated by reference as if fully set forth.

### TECHNICAL FIELD

The present disclosure is directed to circular staplers for use in medical procedures, and more particularly to an anvil connection detection device for circular staplers used in medical procedures.

### BACKGROUND

In certain types of surgical procedures, the use of surgical staples has become the preferred method of joining tissue. Therefore, specially designed surgical staplers have been developed for these applications, which can be referred to as circular staplers in some examples. Circular staplers have become particularly useful for performing an anastomosis procedure, as is known. An anastomosis procedure includes joining sections of intestine of a patient together after a portion of intestine has been removed from the patient. The anastomosis procedure requires re-joining the ends of the two tubular sections together to form a continuous tubular pathway, which is accomplished by the circular stapler. During the surgical procedure, an anvil of the circular stapler must be properly connected to a trocar of the circular stapler to properly perform cutting and stapling of the tissue. If the anvil is not properly attached to the trocar before firing (e.g., cutting and stapling), adverse outcomes and improperly secured tissue may occur. In existing circular staplers, it is difficult for the user (e.g., a surgeon) to determine if the anvil is properly attached to the trocar.

Therefore, there is a need to provide feedback to the user that the anvil is properly attached to the trocar before closing and firing of the circular stapler.

### SUMMARY OF THE DISCLOSURE

According to one aspect, a circular stapler can include a housing with a handle extending from the housing. A shaft can be coupled to and extend from the housing in a different direction than the handle. A stapling head can be coupled to a distal end of the shaft, the stapling head being configured to cut tissue and dispense one or more staples into tissue. An adjustment knob can be rotatably coupled to the housing, the adjustment knob being coupled to a first end of a pushrod extending through the housing and the shaft. A trocar can be coupled to a second end of the pushrod, the trocar being positioned within and axially translatable relative to the stapling head. An anvil can be removably coupled to a distal end of the trocar. A feedback system can be disposed within the stapling head. The feedback system is configured to detect connection of the anvil to the trocar before retraction of the trocar into the stapling head. The feedback system can be a lock that is pivotally connected to the trocar.

In one aspect, the lock is connected to an outer radial surface of the trocar.

In one aspect, the lock prevents retraction of the trocar into the stapling head when the anvil is disconnected from the trocar.

In one aspect, the lock is a spring biased lock that extends radially outwards from the trocar upon the trocar extending a specified distance out from the stapling head.

According to another aspect, a circular stapler can include a housing with a handle extending from the housing. A shaft can be coupled to and extend from the housing in a different direction than the handle. A stapling head can be coupled to a distal end of the shaft, the stapling head being configured to cut tissue and dispense one or more staples into tissue. An adjustment knob can be rotatably coupled to the housing, the adjustment knob being coupled to a first end of a pushrod extending through the housing and the shaft. A trocar can be coupled to a second end of the pushrod, the trocar being positioned within and axially translatable relative to the stapling head. An anvil can be removably coupled to a distal end of the trocar. A feedback system can be disposed within the housing or the stapling head, and the feedback system can include at least one sensor configured to detect connection of the anvil to the trocar.

In one aspect, detection of the connection of the anvil to the trocar comprises continuously monitoring one or more of audio signals, frequency signals or ranges, vibration signals, acceleration signals, friction signals, and torque signals.

In one aspect, the one or more audio signals, frequency signals or ranges, vibration signals, acceleration signals, friction signals, and torque signals being lower than a predetermined threshold value indicates connection of the anvil to the trocar.

In one aspect, the one or more audio signals, frequency signals or ranges, vibration signals, acceleration signals, friction signals, and torque signals being greater than a predetermined threshold value indicates nonconnection of the anvil to the trocar.

In one aspect, the detection of the anvil connection to the trocar comprises one or more mechanical features on the anvil or the trocar which generates a signature signal which is compared against expected signals to confirm anvil connection to the trocar.

In one aspect, the at least one sensor is a friction sensor.

In one aspect, the at least one sensor is an accelerometer.

In one aspect, the at least one sensor is a microphone.

In one aspect, the at least one sensor is a piezo-sensing element.

In one aspect, the at least one sensor is a torque sensor.

In one aspect, the at least one sensor is an electrical switch.

In one aspect, the trocar includes an undulating outer surface along an axial length of the trocar, and wherein the electrical switch contacts the undulating outer surface of the trocar.

According to yet another aspect, a circular stapler can include a housing with a handle extending from the housing. A shaft can be coupled to and extend from the housing in a different direction than the handle. A stapling head can be coupled to a distal end of the shaft, the stapling head being configured to cut tissue and dispense one or more staples into tissue. An adjustment knob can be rotatably coupled to the housing, the adjustment knob being coupled to a first end of a pushrod extending through the housing and the shaft. A trocar can be coupled to a second end of the pushrod, the trocar being positioned within and axially translatable relative to the stapling head. An anvil can be removably coupled to a distal end of the trocar. A feedback system can be disposed within the housing or the stapling head, the feedback system being configured to detect connection of the anvil to the trocar before retraction of the trocar into the stapling head. Detection of the connection of the anvil to the trocar can include comparing a torque response of the trocar against a threshold torque value, which torque response is based on a predetermined sequence of motions of the trocar.

In one aspect, the torque response being lower than a predetermined threshold value indicates connection of the anvil to the trocar.

In one aspect, the torque response being greater than a predetermined threshold value indicates nonconnection of the anvil to the trocar.

In one aspect, the predetermined sequence of motions comprises a partial rotation of the trocar in at least one of a first direction and a second direction.

### BRIEF DESCRIPTION OF THE DRAWINGS

The foregoing Summary as well as the following Detailed Description will be best understood when read in conjunction with the appended drawings, which illustrate exemplary embodiments. In the drawings:

FIG. 1 is a perspective view of an exemplary embodiment of a circular stapler according to the present disclosure.

FIG. 2 is a partial side-view of the circular stapler of FIG. 1.

FIG. 3 is a magnified perspective view of a distal end of the circular stapler of FIG. 1, illustrating an anvil separated from a trocar of the circular stapler.

FIG. 4 is a side view of an embodiment of a feedback system of the circular stapler with the anvil separated from the trocar.

FIG. 5 is a side cross-sectional view of the feedback system of FIG. 4 with the trocar in an extended orientation.

FIG. 6 is a side cross-sectional view of the feedback system of FIG. 4, with the anvil attached to the trocar in the extended orientation.

FIG. 7 is a partial side cross-sectional view of another embodiment of a feedback system of the circular stapler with the anvil attached to the trocar.

FIG. 8 is a partial side cross-sectional view of the feedback system of FIG. 7, illustrating only the trocar.

FIG. 9 is a perspective view of a friction element that can be used in the feedback system of FIGS. 7-8.

FIG. 10 is a partial side cross-sectional view of the feedback system of FIGS. 7-9, with the friction element illustrated in a compressed orientation.

FIG. 11 is a perspective view of the trocar of the feedback system of FIGS. 7-10 with the friction element removed.

FIG. 12A is a perspective view of an alternative friction element that can be used in the feedback system of FIGS. 7-11.

FIG. 12B is a perspective view of another alternative friction element that can be used in the feedback system of FIGS. 7-11.

FIG. 12C is a perspective view of yet another alternative friction element that can be used in the feedback system of FIGS. 7-11.

FIG. 13 is a perspective view of another embodiment of a feedback system of the circular stapler, illustrating only the trocar with the anvil removed.

FIG. 14 is a side cross-sectional view of the feedback system of FIG. 13 with the anvil attached to the trocar.

FIG. 15 is a side cross-sectional view of the feedback system of FIGS. 13-14 including a spring feature contacting the trocar.

FIG. 16 is a side cross-sectional view of the feedback system of FIGS. 13-14 including a switch contacting the trocar.

FIG. 17 is a side schematic-view of another embodiment of a feedback system of the circular stapler including an accelerometer.

FIG. 18 is a side schematic-view of another embodiment of a feedback system of the circular stapler including a microphone.

FIG. 19 is a side schematic-view of another embodiment of a feedback system of the circular stapler including a piezo-sensing element.

FIG. 20 is a side cross-sectional view of another embodiment of a feedback system of the circular stapler including a microphone and/or an accelerometer positioned adjacent the trocar and anvil.

FIG. 21 is a side view of another embodiment of a feedback system of the circular stapler including an electronic verification system.

FIG. 22 is a side view of the feedback system of FIG. 21 in use or operation.

FIG. 23 is an acceleration-time graph illustrating an acceleration curve characteristic during attachment of the anvil to the trocar, with an acceleration threshold value identified.

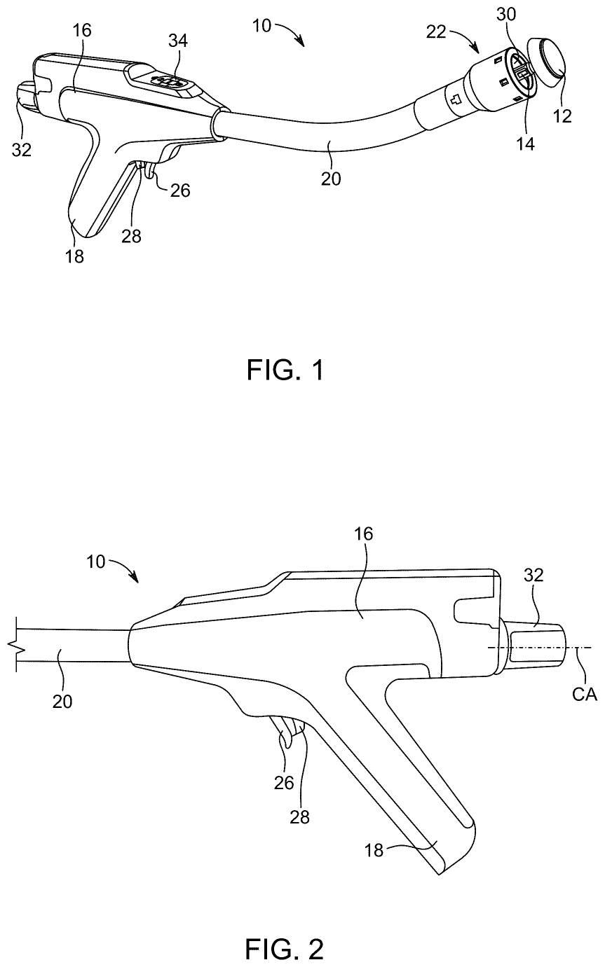

### DETAILED DESCRIPTION

Certain terminology is used in the following description for convenience only and is not limiting. The words “front”, “rear”, “upper”, and “lower” designate directions in the drawings to which reference is made. The words “inwardly” and “outwardly” refer to directions towards and away from parts referenced in the drawings. “Axially” refers to a direction along the axis of a shaft or other cylindrically shaped component. A reference to a list of items that are cited as “at least one of a, b, or c” (where a, b, and c represent the items being listed) means any single one of the items a, b, or c, or combinations thereof. The terms “generally” and “approximately” are to be construed as within 10% of a stated value or ratio, unless otherwise noted. Additionally, the terms “proximal” and “distal” are used with reference to a handle portion of a circular stapler disclosed herein. The term “proximal” referring to the portion closest to the handle portion and the term “distal” referring to the portion located away from the handle portion in a direction of a tip of the circular stapler. The terminology includes the words specifically noted above, derivatives thereof, and words of similar import.

The circular stapler 10 includes a housing 16 with a handle 18 extending downwards and away from the housing 16. The handle 18 may be grasped by a user (e.g., a surgeon) while using the circular stapler 10. It is to be understood that the terms “user” and “surgeon” may be used interchangeably throughout the present disclosure, with the intended meaning being the person holding and operating the circular stapler 10. Additionally, it is to be understood that the “user” could be any person that is applicable for the purpose of this disclosure. In other embodiments, the circular stapler 10 can be removably coupled to or integrated into a robotics system in which a robotic arm supports and operates the circular stapler 10 based on the surgeon's instructions/guidance through a remotely operated graphical user interface (GUI) or other controller communicatively coupled to the robotic arm/system.

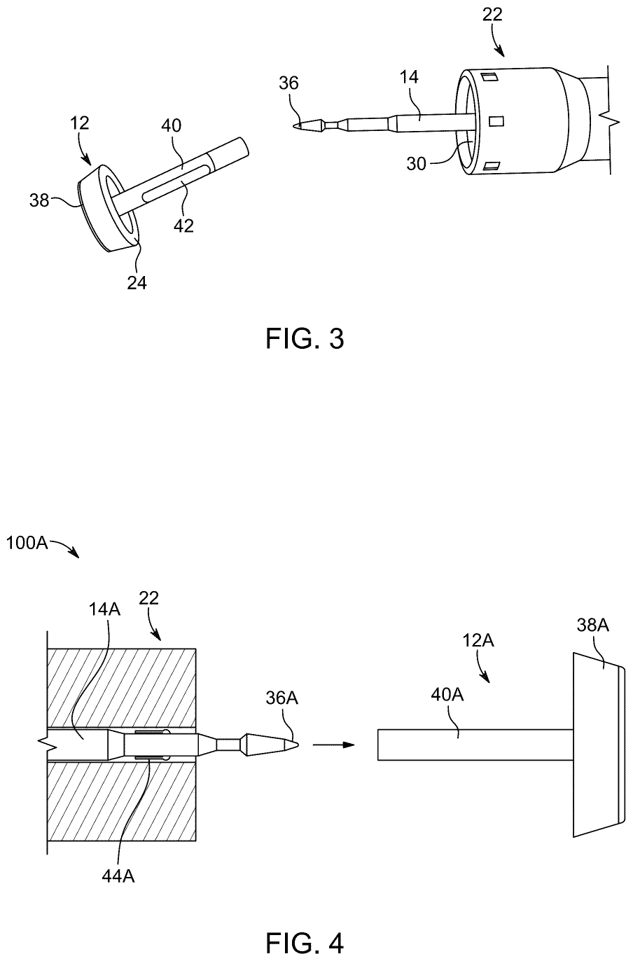

A shaft 20 extends outwards from the housing 16, with the shaft 20 having the shape of an elongated tube with a circular cross-section. Additionally, the shaft 20 may include a slight bend or curvature, such that the shaft 20 is not a straight tube. A stapling head 22 (see FIG. 3 for clarity) is positioned at a distal end of the shaft 20, with the distal end of the shaft 20 being the end furthest from the housing 16 of the circular stapler 10. A trocar 14 is positioned within and axially aligned with the stapling head 22, and the trocar 14 is axially translatable relative to the stapling head 22. In other words, the trocar 14 can translate axially inwardly and outwardly relative to the stapling head 22, such that the trocar 14 can extend axially outwards of the stapling head 22 (see FIG. 3) and the trocar 14 can be positioned fully within and surrounded by the stapling head 22. An anvil 12 is removably coupled to the trocar 14, such that the anvil 12 can be connected to and removed from the trocar 14, discussed further below. When the anvil 12 is coupled to the trocar 14, the anvil 12 is axially aligned with the trocar 14 and therefore also axially aligned with the stapling head 22. Additionally, when the anvil 12 is coupled to the trocar 14, the anvil 12 is also axially translatable relative to the stapling head 22. The anvil 12 includes a staple forming surface 24 that is configured to engage with a distal face or surface of the stapling head 22, discussed further below.

A trigger 26 is pivotally coupled to the housing 16 adjacent the handle 18, with the trigger 26 being pivotal relative to the housing 16. Actuation of the trigger 26 causes firing or operation of the circular stapler 10, discussed further below. A safety 28 is coupled to the housing 16 and positioned adjacent the trigger 26, between the trigger 26 and the handle 18. The safety 28 is pivotal relative to the housing 16, and the safety 28 is configured to prevent the trigger 26 from being actuated when in the locked or safe position, and the safety allows the trigger 26 to be actuated when in the released or fire position. The released or fire position being the position in which the safety 28 is pivoted away from the trigger 26, allowing the trigger 26 to be actuated which fires or operates the circular stapler 10. When the trigger 26 is actuated, an internal drive system (not shown) of the circular stapler 10 operates within the shaft 20 to cause staples to be dispensed from the stapling head 22 into forming contact with the staple forming surface 24. Simultaneously, a knife 30 (see FIG. 3), that is operably supported within the stapling head 22, translates axially outwards from the stapling head 22 to cut tissue held within a circumference of the stapling head 22, as will be explained in further detail hereinafter.

The circular stapler 10 also includes an adjustment knob 32 coupled to a proximal end of the housing 16 that is furthest from the stapling head 22 of the circular stapler 10. The adjustment knob 32 is configured to rotate both clockwise and counterclockwise about a central axis (CA) of the adjustment knob 32. The adjustment knob 32 is connected to the internal drive system (not shown) within the housing 16 of the circular stapler 10, and the distal end of the internal drive system is connected to the trocar 14 adjacent and within the stapling head 22. Rotation of the adjustment knob 32 causes actuation of the internal drive system within the housing 16 and the shaft 20, which in turn causes the trocar 14 to translate axially inwardly and outwardly relative to the stapling head 22. As such, the user rotates the adjustment knob 32 at a proximal end of the circular stapler 10 to control the axial translation and positioning of the trocar 14 relative to the stapling head 22 at the distal end of the circular stapler 10. The circular stapler 10 can also include a display 34 that is positioned on a side of the housing 16, opposite the extending direction of the handle 18. The display 34 provides an indication when the appropriate tissue compression has been achieved, discussed further below.

Referring to FIG. 3, the knife 30 is a circular shaped knife 30 that is positioned within and axially aligned with the stapling head 22. When the trigger 26 is pulled and the internal drive mechanism is actuated (or fired), the knife 30 quickly translates axially outwards from the stapling head 22 and then retracts back into the stapling head 22. The knife 30 is configured to cut tissue that is compressed between the staple forming surface 24 of the anvil 12 and the distal end face or surface of the stapling head 22. The trocar 14 is an elongated metallic shaft with a sharp-pointed distal tip 36 that is configured to cut through tissue. It is to be understood that the trocar 14 can be any surgical instrument with cutting edges at the distal tip 36 for cutting tissue. Additionally, the trocar 14 is shaped and sized to couple to the anvil 12.

The anvil 12 includes an anvil shroud 38 which is a generally circular body portion of the anvil 12. The staple forming surface 24 is formed on the underside of the anvil shroud 38, which underside faces the stapling head 22 when the anvil 12 is coupled to the trocar 14. An anvil shaft 40 extends from the underside of the anvil shroud 38, in a direction axially away from the anvil shroud 38 such that the anvil shaft 40 is axially aligned with the anvil shroud 38. The anvil shaft 40 can have the shape of a hollow or a partially hollow elongated cylinder, which shape is a female mating shape to the male mating shape of the trocar 14. Therefore, the anvil shaft 40 and anvil 12 include complimentary shapes and sizes, such that the anvil 12 can be inserted over the distal tip 36 of the trocar 14 to be coupled to and/or removed from the trocar 14. The anvil 12 can further include at least one retaining clip 42 pivotally coupled to the anvil shaft 40. In some examples, the at least one retaining clip 42 can be a leaf-type spring or other spring component that snaps or latches onto features of the trocar 14 to retain the anvil 12 on the trocar 14. Further, in some examples, the at least one retaining clip 42 can be two retaining clips 42 positioned on opposite sides of the anvil shaft 40.

As discussed, the circular stapler 10 includes the elongated shaft 20 and a distal stapling head 22 with a stapling mechanism mounted to the distal end of the shaft 20. The stapling head 22 can also include a stapling cartridge (not shown) that contains a plurality of staples configured in a concentric circular array. The knife 30 can be concentrically mounted within the stapling cartridge and configured to travel axially within the stapling cartridge. Additionally, the trocar 14 can extend axially from a center of the stapling cartridge, and the trocar 14 is movable relative to the stapling cartridge. The trocar 14 is also adapted to be removably coupled to the anvil 12, as previously discussed. The anvil 12 is configured to form the ends of the staples as they are driven into the staple forming surface 24 of the anvil 12. The distance between a distal face of the staple cartridge and the staple forming surface 24 of the anvil 12 is controlled by adjusting the adjustment knob 32 mounted to the proximal end of the housing 16 of the circular stapler 10. The adjustment knob 32 being configured for controlling the axial movement of the trocar 14. Tissue clamped between the staple cartridge and the staple forming surface 24 of the anvil 12 is simultaneously stapled and cut when the trigger is actuated by the surgeon.

In some examples, when performing an anastomosis using the circular stapler 10, the intestinal tissue is stapled using double rows of staples being placed on either side of the tissue of the intestine to be removed. The adjoining sections of tissue are simultaneously cut as the adjoining sections of tissue are stapled. In such examples, the surgeon typically inserts the anvil 12 into the proximal end of the lumen (intestinal tissue), proximal of the staple line. This is done by inserting the anvil shroud 38 into an entry port cut into the proximal lumen by the surgeon. In some instances, the anvil 12 can be placed transanally, by placing the anvil shroud 38 on the distal end of the circular stapler 10 and inserting the instrument through the rectum. The surgeon then ties the proximal end of the intestine to the anvil shaft 40 using a suture or other conventional tying device.

Next, the surgeon may cut excess tissue adjacent to the tie and the surgeon attaches the anvil 12 to the trocar 14 of the circular stapler 10. The surgeon then closes the gap between the anvil 12 and stapling head 22 by rotating the adjustment knob 32, thereby clamping the proximal and distal ends of the intestine in the gap between the staple forming surface 24 and the distal end face or surface of the stapling head 22. During closing of the gap, the surgeon watches the display 34, which indicates when the correct amount of compression has been achieved based on the tissue thickness. The surgeon next actuates the trigger 26 causing several rows of staples to be driven through both ends of the intestine and formed, thereby joining the ends and forming a tubular pathway. Simultaneously, as the staples are driven and formed, the knife 30 is driven through the intestinal tissue ends, cutting the ends adjacent to the inner row of staples. The surgeon then withdraws the circular stapler 10 from the intestine and the anastomosis procedure is complete.

During the aforementioned stapling and cutting process, it is often difficult for the surgeon to effectively view the area of tissue being cut and stapled. Additionally, during the surgical procedure, the anvil 12 of the circular stapler 10 must be properly connected to the trocar 14 of the circular stapler 10 to properly perform cutting and stapling of the tissue. If the anvil 12 is not properly attached to the trocar 14 before the cutting and stapling, adverse outcomes and improperly secured tissue may occur. In previous circular staplers, it was difficult for the user (e.g., surgeon) to determine if the anvil was properly attached to the trocar. Circular stapler 10 alleviates the aforementioned issues by providing feedback to the user that the anvil 12 is properly attached to the trocar 14 before closing and firing of the circular stapler 10. Several different embodiments of feedback systems are described in detail below, with each feedback system providing an indication to the user that the anvil 12 is properly attached to the trocar 14 before closing and firing of the circular stapler 10.

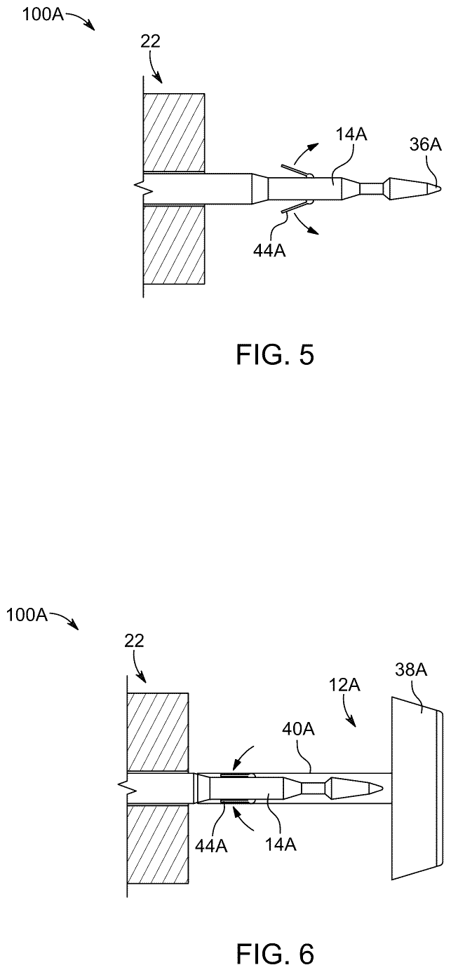

FIG. 4 is a side view of an embodiment of a feedback system 100A of the circular stapler 10 with the anvil 12A separated from the trocar 14A. FIG. 5 is a side cross-sectional view of the feedback system 100A of FIG. 4 with the trocar 14A in an extended orientation. FIG. 6 is a side cross-sectional view of the feedback system 100A of FIG. 4, with the anvil 12A attached to the trocar 14A in the extended orientation. FIGS. 4-6 will be discussed together. The feedback system 100A is configured to provide feedback to the user that the anvil 12A is properly attached to the trocar 14A before closing and firing of the circular stapler 10.

The feedback system 100A includes a trocar 14A with a lock 44A coupled to the trocar 14A. In some examples, the lock 44A can be coupled to exterior side surfaces of the trocar 14A. Further, in some examples, the lock 44A can be a single lock or the lock 44A can comprise a plurality of locks 44A. The lock 44A is a mechanical locking mechanism that is biased outwards by a biasing member, such as a spring, away from a central axis of the trocar 14A. In some examples, the lock 44A can be described as a lockout wing that is biased outwardly by a spring. When the trocar 14A is positioned within the stapling head 22, as illustrated in FIG. 4, the lock 44A is compressed and collapsed against the side surfaces of the trocar 14A. Then when the trocar 14A is translated axially out of the stapling head 22 a far enough distance such that the lock 44A is fully exposed, as illustrated in FIG. 5, the lock 44A is biased by the biasing member outwards away from the central axis of the trocar 14A. With the lock 44A in the extended/open configuration, the trocar 14A is prevented from translating axially back into the stapling head 22. Specifically, if the trocar 14A is translated axially back into the stapling head 22, the lock 44A will contact a distal end surface of the stapling head 22, preventing the trocar 14A from retracting all the way back into the stapling head 22.

To retract the trocar 14A back into the stapling head 22, the anvil 12A must first be properly attached to the trocar 14A. As illustrated in FIG. 6, when the anvil 12A is axially inserted onto and secured to the trocar 14A, the interior surfaces of the anvil shaft 40A contact the lock 44A and force the lock 44A to pivot inwards towards the trocar 14A until the lock 44A is pressed against side surfaces of the trocar 14A. In some examples, the anvil 12A can be axially translated onto the trocar 14A far enough such that the lock 44A is positioned fully within the anvil shaft 40A. With the trocar 14A positioned within the anvil shaft 40A and the lock 44A collapsed, the connected anvil 12A and trocar 14A can be translated back into the stapling head 22 without interference. If the trocar 14A is inserted into the anvil shaft 40A, but the secured connection between the components is not complete, the anvil 12A will slip off the end of the trocar 14A during retraction of the trocar 14A. In turn, this will cause the lock 44A to extend outwards and prevent further retraction of the trocar 14A into the stapling head 22. In this example, lock 44A is a feedback system 100A that provides feedback to the user that the anvil 12A is properly attached to the trocar 14A before closing and firing of the circular stapler 10. If the anvil 12A is not properly attached, the user will not be able to fully retract the trocar 14A.

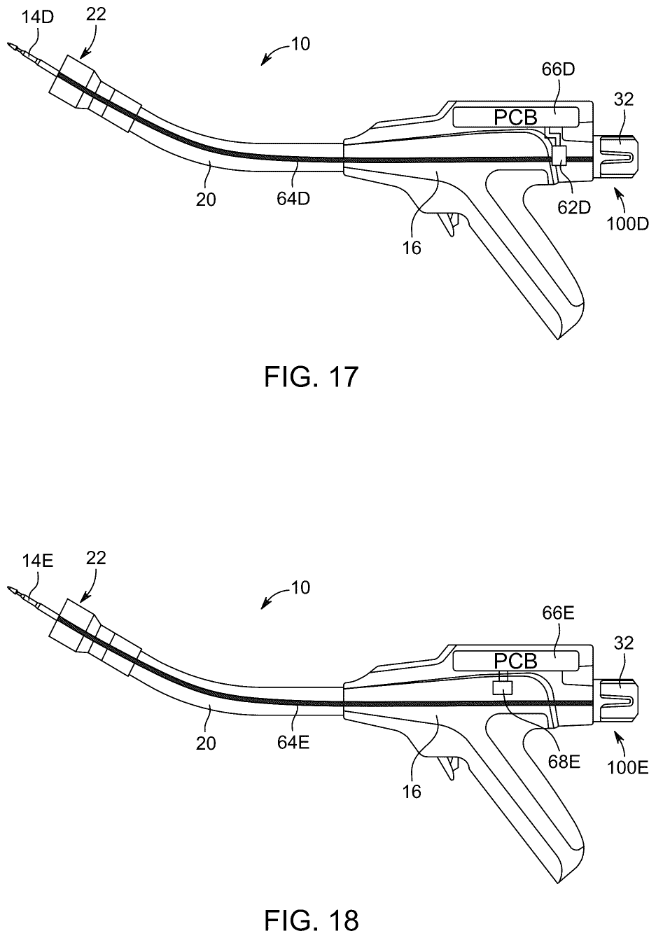

In some examples, the circular stapler 10 can include a printed circuit board, battery, sensors, and other electronics to monitor aspects of operating the circular stapler 10. In such examples, if the anvil 12A is not attached and the user attempts to retract the trocar 14A, the lock 44A resists the retraction which causes an increased torque and/or electrical current within the circular stapler 10. The circular stapler 10 can include processing components that analyze changes in a retraction torque value and/or an increase in electrical current being used, and then compare those values to a predetermined threshold value in order to detect whether the anvil 12A is properly attached to the trocar 14A. In some examples, as illustrated in FIGS. 17-19, the processing components and the sensor can be located within the housing 16 of the circular stapler 10 (e.g. accelerometer 62D and printed circuit board 66D in FIG. 17). If the compared sensed values exceeds the threshold value, an alarm and other indication on the display 34 can notify the surgeon that the anvil 12A is not properly attached to the trocar 14A. Other factors such as speed and displacement of the trocar 14A can also be measured, and the one or more parameters that are detected can then be compared to predetermined threshold values. It is to be understood that the predetermined threshold values can be obtained through mathematical computation, experimental testing, or other techniques known in the art.

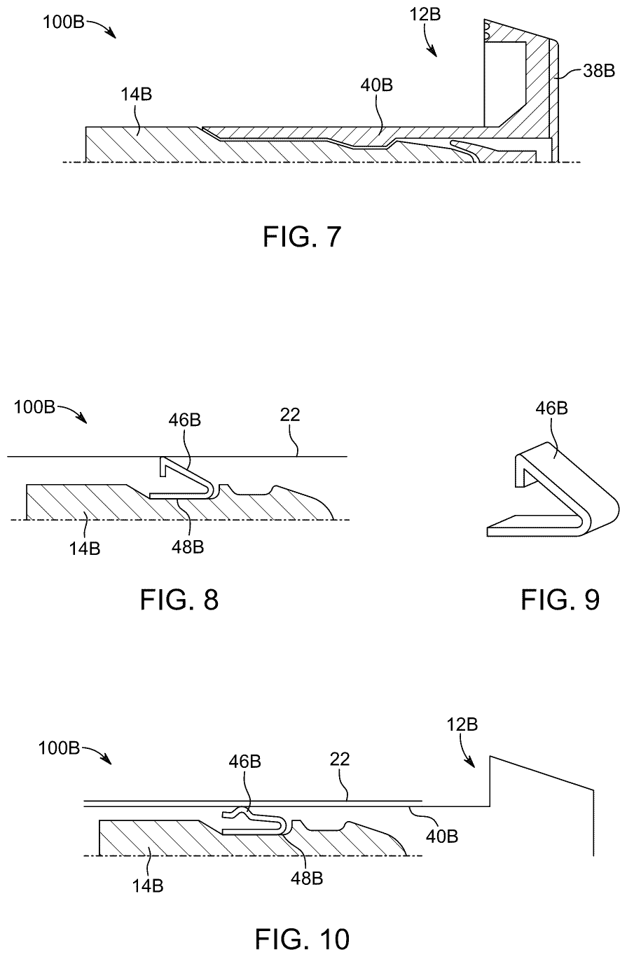

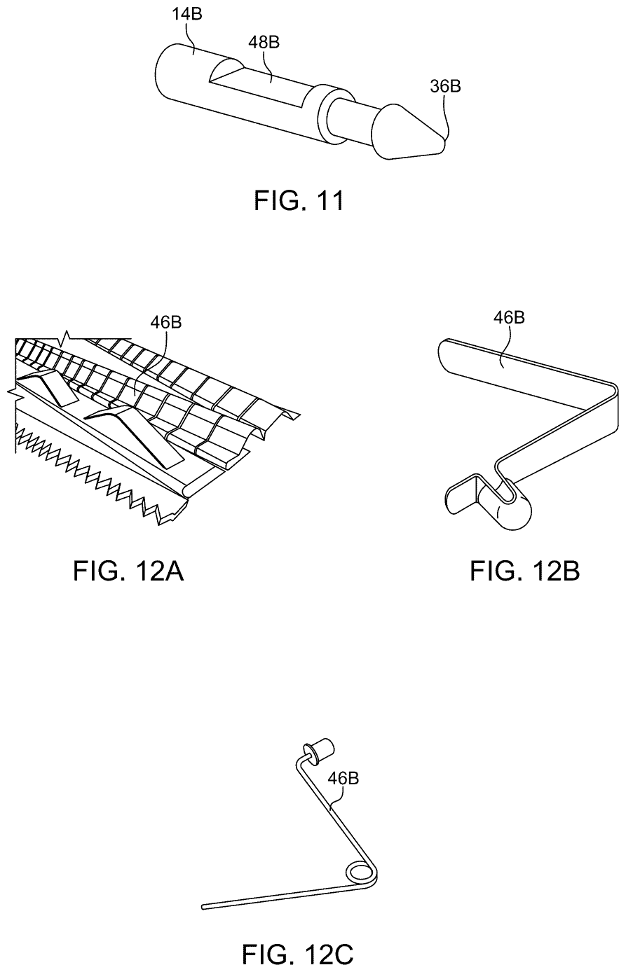

FIG. 7 is a partial side cross-sectional view of another embodiment of a feedback system 100B of the circular stapler 10 with the anvil 12B attached to the trocar 14B. FIG. 8 is a partial side cross-sectional view of the feedback system 100B of FIG. 7, illustrating the trocar 14B. FIG. 9 is a perspective view of a friction element 46B that can be used in the feedback system 100B of FIGS. 7-8. FIG. 10 is a partial side cross-sectional view of the feedback system 100B of FIGS. 7-9, with the friction element 46B illustrated in a compressed orientation. FIG. 11 is a perspective view of the trocar 14B of the feedback system 100B with the friction element 46B removed. FIGS. 12A-12C are perspective views of alternative friction elements 46B that can be used in the feedback system 100B. FIGS. 7-12C will be discussed together. The feedback system 100B is configured to provide feedback to the surgeon that the anvil 12B is properly attached to the trocar 14B before closing and firing of the circular stapler 10.

The feedback system 100B includes a trocar 14B with a friction element 46B coupled to the trocar 14B. Referring to FIG. 11, the trocar 14B of the feedback system 100B includes a contact surface 48B that is a cutout that extends from an outer circumference of the trocar 14B towards the central axis of the trocar 14B. In some examples, as illustrated, the contact surface 48B can be a flat surface that extends generally parallel to the central axis of the trocar 14B. The contact surface 48B is a surface of the trocar 14B that is configured to receive and secure the friction element 46B. FIGS. 12A-12C are perspective views of various friction elements 46B that can be used in the feedback system 100B. The friction element 46B can be a contact spring (FIG. 12A), a biased pin (FIGS. 12B-12C), or any other component that is biased outwards away from the central axis of the trocar 14B to create friction between the friction element 46B and inner sidewalls of the stapling head 22 in which the trocar 14B is positioned within.

Referring to FIGS. 8-10, the friction element 46B is positioned within and coupled to the contact surface 48B of the trocar 14B. The friction element 46B can be coupled to the contact surface 48B through an adhesive, fastener, or other known fastening technique. As illustrated in FIG. 8, when the trocar 14B is not positioned within the anvil shaft 40B of the anvil 12B, the friction element 46B is biased outwards away from the central axis of the trocar 14B. Additionally, when the trocar 14B is not positioned within the anvil shaft 40B and the trocar 14B is retracted into the stapling head 22, the friction element 46B contacts an inner surface of the stapling head 22 in which the trocar 14B translates axially inwardly and outwardly. The contact between the friction element 46B and the inner surfaces of the stapling head 22 creates and increased friction between the components which makes it more difficult to retract the trocar 14B into the stapling head 22.

The circular stapler 10 can include a printed circuit board, battery, sensors, and other electronics to monitor aspects of operating the circular stapler 10. Therefore, the increased friction due to the friction element 46B can be actively sensed and monitored during the retraction process of the trocar 14B. When a distally extended trocar 14B is retracted along a specified amount of travel without the anvil 12B properly attached, the friction sensed by the circular stapler 10 will be above a predetermined threshold load limit. In some examples, as illustrated in FIGS. 17-19, the processing components and the sensor can be located within the housing 16 of the circular stapler 10 (e.g. accelerometer 62D and printed circuit board 66D in FIG. 17). The circular stapler 10 can then communicate to the user that the anvil 12B is not attached, based on the sensed increase in loads and/or retraction forces. The communication can be an audible alarm and/or the communication can be a light or message displayed on the display 34 of the circular stapler 10.

In contrast, as schematically illustrated in FIG. 10, when the anvil 12B is properly attached to the trocar 14B, the friction element 46B is compressed within the interior of the anvil shaft 40B. Then, when the coupled anvil 12B and trocar 14B are retracted into the stapling head 22, the friction element 46B does not contact the stapling head 22 and the sensed friction value will be below the predetermined threshold load limit, which indicates to the user that the anvil 12B is properly attached to the trocar 14B. In some examples, the circular stapler 10 can detect rate of change in torques, electrical currents, speed of trocar 14B retraction, and displacement of the trocar 14B, among other options not specifically listed. The feedback system 100B provides another feedback solution that communicates to the user that the anvil 12B is properly attached to the trocar 14B before closing and firing of the circular stapler 10.

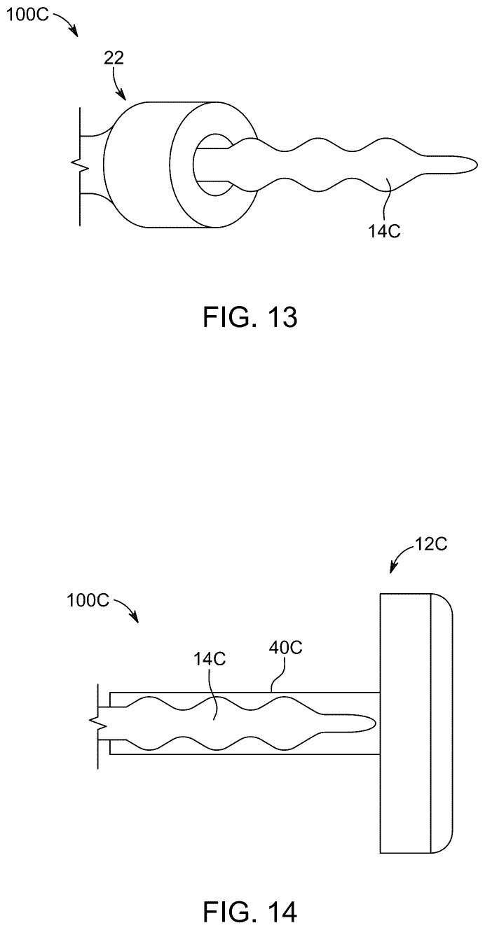

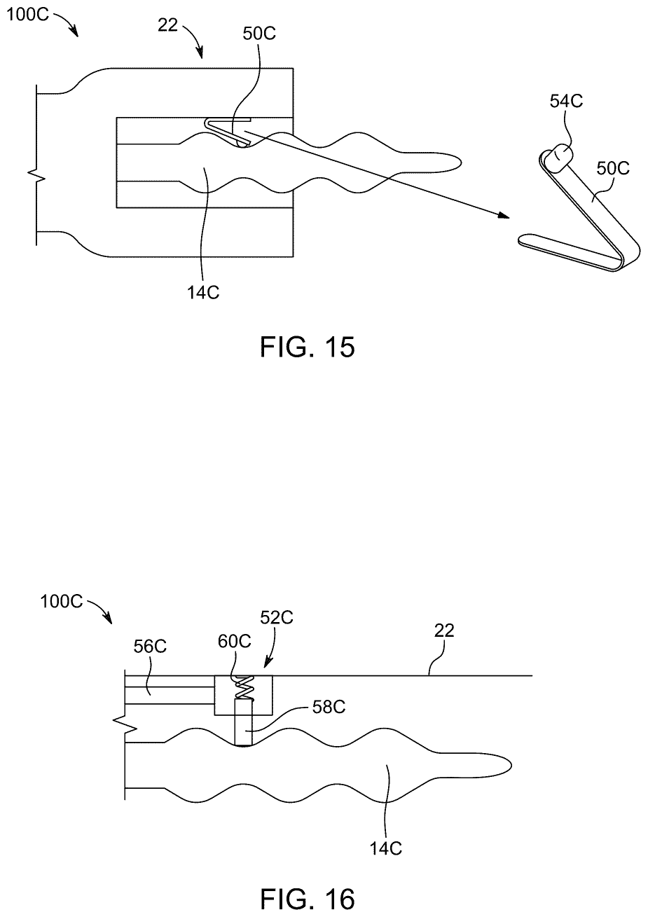

FIG. 13 is a perspective view of another embodiment of a feedback system 100C of the circular stapler 10, illustrating only the trocar 14C extending from the stapling head 22 with the anvil 12C removed. FIG. 14 is a side cross-sectional view of the feedback system 100C of FIG. 13 with the anvil 12C attached to the trocar 14C. FIG. 15 is a side cross-sectional view of the feedback system 100C including a spring feature 50C contacting the trocar XC. FIG. 16 is a side cross-sectional view of an alternative feedback system 100C including a switch 52C contacting the trocar 14C. FIGS. 13-16 will be discussed together. The feedback system 100C is configured to provide feedback to the surgeon that the anvil 12C is properly attached to the trocar 14C before closing and firing of the circular stapler 10.

Specifically, in the feedback system 100C the trocar 14C includes an undulating outer surface, which can be described as including smooth bumps, ribs, or wave-like features extending axially along the outer surface of the trocar 14C. Therefore, the trocar 14C does not include a flat and smooth outer surface like previous trocars, but rather the trocar 14C includes an undulating outer surface. As illustrated in FIG. 13, the trocar 14C with the undulating outer surface is axially translatable inwardly and outwardly relative to the stapling head 22, similar to previous embodiments. Additionally, as illustrated in FIG. 14, the trocar 14C with the undulating outer surface is shaped and sized to fit within the anvil shaft 40C of the anvil 12C, such that the anvil 12C can be coupled to the trocar 14C. Further, when the trocar 14C with the undulating outer surface is coupled to the anvil 12, the undulating outer surface of the trocar 14C is fully covered by the anvil shaft 40C.

As illustrated in FIG. 15, which is a cross-sectional view of the feedback system 100C including a spring feature 50C positioned within the stapling head 22 that is configured to contact the trocar 14C. Specifically, the spring feature 50C is fixedly coupled with an interior of the stapling head 22, and the spring feature 50C is biased inwardly towards a central axis of the stapling head 22. The spring feature 50C can be coupled at a first end to the inner surface of the stapling head 22 and the spring feature 50C can include a knob 54C positioned at the opposite end of the spring feature 50C that is not coupled to the stapling head 22. The knob 54C is configured to contact and engage the undulating outer surface of the trocar 14C during axially translation of the trocar 14C. Additionally, the spring feature 50C can be electrically connected to a printed circuit board or other electrical processing components through an electrical wire (not shown) that extends through the housing 16 between the components.

In use, the spring feature 50C of the feedback system 100C is configured to translate radially in and out along the outer undulating surface of the trocar 14C during axial translation of the trocar 14C, when the anvil 12C is not attached to the trocar 14C. The radially movement of the spring feature 50C is transferred as electrical data/signals through the electrical wire to the printed circuit board or other electrical processing components of the circular stapler 10. The circular stapler 10 can then process the data to determine that the spring feature 50C is moving radially, which indicates that the anvil 12C is not attached to the trocar 14C. The circular stapler 10 can then communicate to the surgeon that the anvil 12C is not properly attached to the trocar 14C. When the anvil 12C is attached to the trocar 14C (see FIG. 14), the spring feature 50C will not move radially because the spring feature 50C contacts the smooth outer surface of the anvil shaft 40C of the anvil 12C. Since the spring feature 50C is not transferring data indicating radial movement, the circular stapler 10 can indicate to the surgeon that the anvil 12C is properly attached to the trocar 14C.

FIG. 16 is a side cross-sectional view of an alternative feedback system 100C including a switch 52C contacting the trocar 14C. The feedback system 100C in FIG. 16 is substantially similar to the feedback system 100C of FIG. 15, with the only difference being that the spring feature 50C is replaced with a switch 52C. The switch 52C is fixedly coupled within an interior of the stapling head 22C, and the switch 52C is electrically coupled through an electrical wire 56C to the printed circuit board or other electrical processing components of the circular stapler 10. The switch 52 includes a switch button 58C that is biased inwardly towards a center of the stapling head 22C by a switch spring 60C. The switch button 58C is configured to contact and engage the undulating outer surface of the trocar 14C during axially translation of the trocar 14C.

When the switch button 58C and switch spring 60C are compressed due to a high point of the undulating surface, the switch 52 turns on and sends an on-signal to the printed circuit board or other electrical processing components of the circular stapler 10. When the switch button 58C and switch spring 60C are uncompressed due to a low point of the undulating surface, the switch 52 turns off and sends an off-signal to the printed circuit board or other electrical processing components of the circular stapler 10. Therefore, the printed circuit board or other electrical processing components of the circular stapler 10 can process the data, and when the data indicates frequently turning on and off of the switch 52C, this is an indication that the anvil 12C is not attached to the trocar 14C. When the anvil 12C is attached to the trocar 14C, the signal sent by the switch 52C will be constantly in the on-position, indicating that the anvil 12C is properly coupled to the trocar 14C. The feedback system 100C provides another feedback solution that communicates to the surgeon that the anvil 12C is properly attached to the trocar 14C before closing and firing of the circular stapler 10.

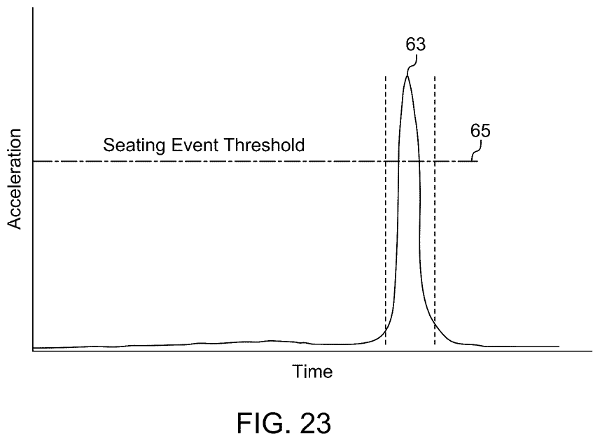

FIG. 17 is a side schematic-view of another embodiment of a feedback system 100D of the circular stapler 10 including an accelerometer 62D. FIG. 23 is an acceleration-time graph illustrating an acceleration curve 63 characteristic during attachment of the anvil 12C to the trocar 14C, with an acceleration threshold value 65 identified. In some examples, the acceleration threshold value 65 can be between +/−1 g (9.8 m/s²) to +/−250 g (2,450 m/s²). The feedback system 100D is configured to provide feedback to the user that the anvil 12D (not shown) is properly attached to the trocar 14D before closing and firing of the circular stapler 10. The feedback system 100D utilizes an accelerometer 62D on an end of a pushrod 64D that is coupled between the trocar 14D and the adjusting knob 54D, within the shaft 20 and the housing 16. The pushrod 64D is configured to axially translate the trocar 14D based on rotation of the adjusting knob 54D, as previously discussed. The accelerometer 62D is coupled to the pushrod 64D at a proximal end of the circular stapler 10, and the accelerometer 62D is configured to detect a force or acceleration curve characteristic (see FIG. 23) when the anvil 12D is successfully and properly attached to the trocar 14D, discussed further below.

The accelerometer 62D and a printed circuit board 66D (i.e., a processor) are communicatively connected for data transfer, and both are located within the housing 16 of the circular stapler 10. The accelerometer 62D is used to detect an acceleration curve characteristic (see FIG. 23) produced by the attachment of the anvil 12D to the trocar 14D. Specifically, since the pushrod 64D is used to connect the trocar 14D to the anvil 12D, a characteristic force is transferred across the pushrod 64D. As the trocar 14D seats into the anvil 12D, the force experiences a sudden dip, which in turn allows for a rapid acceleration of the pushrod 64D. If the printed circuit board 66D and the accelerometer 62D detect that the acceleration surpasses a predetermined acceleration threshold value 65, then falls back below the predetermined threshold value within a set period of time (for example 5 milli-seconds), then a seating event is recorded which indicates a proper connection between the anvil 12D and the trocar 14D (see FIG. 17 without the anvil 12D illustrated).

In some examples, the accelerometer 62D can be multi-axis, such that when the accelerometer 62D records component accelerations (x, y, z, yaw, pitch, roll), the component of those accelerations reflected upon the axis of the trocar 14D is calculated. The reflected acceleration is then used to determine proper anvil 12D seating on the trocar 14D. In other examples, the trocar 14D axis relative to the accelerometer 62D is determined by providing a known acceleration about the actual trocar 14D in manufacture, recording the accelerometer's response, and then back calculating the orientation necessary to predict the known, provided input acceleration. The back calculating and comparing can as be used to determine proper anvil 12D seating on the trocar 14D, as will be appreciated by those skilled in the art. The feedback system 100D provides another feedback solution that communicates to the surgeon that the anvil 12D is properly attached to the trocar 14D before closing and firing of the circular stapler 10.

FIG. 18 is a side schematic-view of another embodiment of a feedback system 100E of the circular stapler 10 including a microphone 68E. The feedback system 100E is configured to provide feedback to the surgeon that the anvil 12E (not shown) is properly attached to the trocar 14E before closing and firing of the circular stapler 10. The feedback system 100E uses a surface microphone 68E or other sound-based sensing device in the proximal end of the housing 16 to detect the “click” issued from the anvil 12E during successful anvil 12E attachment to the trocar 14E. Specifically, the microphone 68E can be positioned adjacent a proximal end of a pushrod 64E that extends from the adjusting knob 54E to the trocar 14E. Therefore, when the anvil 12E is attached to the trocar 14E, the vibration created by the “click” during the snap connection will be transferred through the pushrod 64E and record or identified by the microphone 68E.

A printed circuit board 66E (i.e., a processor) is communicatively coupled to the microphone 68E, and the microphone 68E and the printed circuit board 66E are coupled within the housing 16 of the circular stapler 10. The printed circuit board 66E is configured to sample the analog output of the microphone 68E and conduct fast fourier transform (FFT) to isolate pre-defined frequencies. If specified and identified frequencies are observed, the printed circuit board 66E communicates and outputs a notification that the anvil 12E is attached to the trocar 14E. The notification to the user can be an audible notification and/or a light or message on the display 34, among other options not specifically listed.

In another embodiment, the anvil 12E can be modified to have a first and second click during the connection process in a first and second frequency range, respectively. The printed circuit board 66E continuously scans and, if the first and then the second click is observed, the printed circuit board 66E communicates and outputs a notification that the anvil 12E is attached to the trocar 14E. Further, if the printed circuit board 66E identifies that the second and then the first click is observed, the printed circuit board 66E communicates and outputs a notification that the anvil 12E is detached from the trocar 14E. Therefore, the microphone 68E and the printed circuit board 66E can identify and distinguish between the frequency of the clicks or sounds produced between the anvil 12E and the trocar 14E to determine when the anvil 12E is attached to the trocar 14E, and when the anvil 12E is removed or detached from the trocar 14E. In some examples, detection of the connection of the anvil to the trocar comprises continuously monitoring frequency signals or ranges. The one or more frequency signals or ranges being lower than a predetermined threshold value can indicate connection of the anvil to the trocar, and the one or more frequency signals or ranges being greater than a predetermined threshold value can indicate nonconnection of the anvil to the trocar.

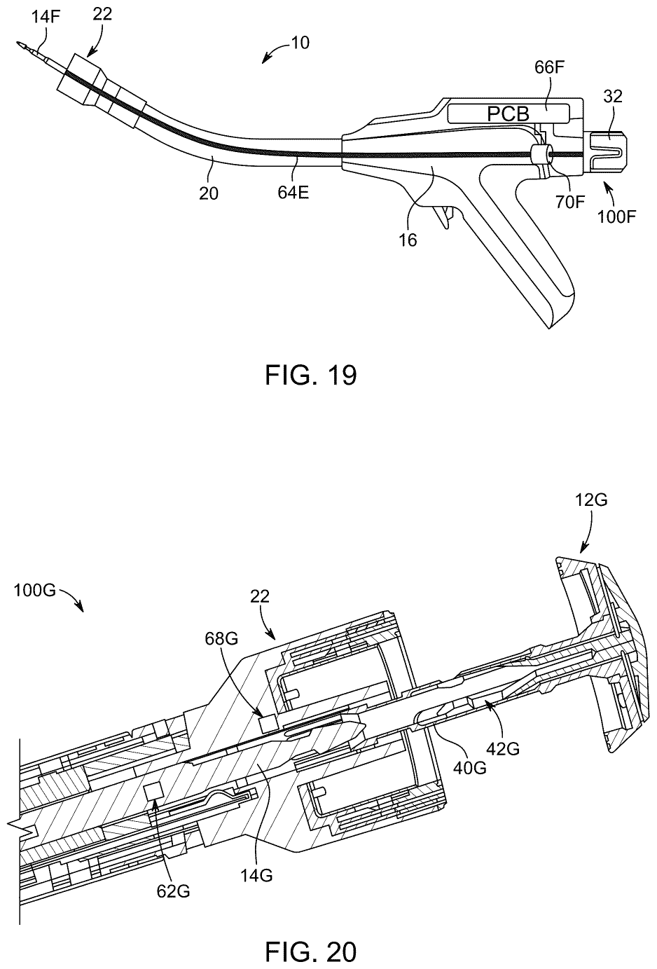

FIG. 19 is a side schematic-view of another embodiment of a feedback system 100F of the circular stapler 10 including a piezo-sensing element 70F. The feedback system 100F is configured to provide feedback to the surgeon that the anvil 12F (not shown) is properly attached to the trocar 14F before closing and firing of the circular stapler 10. In the feedback system 100F, a printed circuit board 66F (i.e., a processor) and the piezo-sensing element 70F are communicatively coupled and both are coupled and positioned within the housing 16. Further, the piezo-sensing element 70F is rigidly coupled to the pushrod 64F, which is coupled to the trocar 14F of the circular stapler 10.

The piezo-sensing element 70F is configured to convert vibrations along the pushrod 64F to an analog voltage output. The more intense vibrations sensed by the piezo-sensing element 70F, the higher voltage output produced by the piezo-sensing element 70F and sent to the printed circuit board 66F. The printed circuit board 66F is configured to sample the analog voltage that is output and sent by the piezo-sensing element 70F. When a voltage output exceeds a predetermined voltage threshold value, the printed circuit board 66F communicates and outputs a notification that the anvil 12F is attached to the trocar 14F. The notification to the user can be an audible notification and/or a light or message on the display 34, among other options not specifically listed. The feedback system 100F provides another feedback solution that communicates to the surgeon that the anvil 12F is properly attached to the trocar 14F before closing and firing of the circular stapler 10.

FIG. 20 is a side cross-sectional view of another embodiment of a feedback system 100G of the circular stapler 10 including a microphone 68G and/or an accelerometer 62G positioned adjacent the trocar 14G and the anvil 12G. The embodiment illustrated in FIG. 20 is similar to the embodiments illustrated in FIGS. 17-18, except that the microphone 68G and the accelerometer 62G in the embodiment of FIG. 20 are positioned at the distal end of the circular stapler 10 rather than the proximal end of the circular stapler 10 (FIGS. 17-18). Therefore, the disclosure regarding FIGS. 17-18 is to be understood as equally applying to the embodiment of FIG. 20 unless otherwise stated, with the main difference being the positioning of the microphone 68G and the accelerometer 62G within the circular stapler 10. The feedback system 100G is configured to provide feedback to the surgeon that the anvil 12G is properly attached to the trocar 14G before closing and firing of the circular stapler 10.

In the feedback system 100G, the microphone 68G is positioned in proximity to the interface of the anvil 12G and the trocar 14G. The microphone 68G is electrically wired to a printed circuit board (not shown) of the circular stapler 10 within the shaft 20 and the housing 16. When the anvil 12G is attached to the trocar 14G, the attachment “click” is heard and identified by the microphone 68G which is in close proximity to the attachment location. The algorithms within the printed circuit board analyze the audio signal and detect the “click” of the anvil 12G attachment to the trocar 14G. Additionally, the printed circuit board includes algorithms such that the printed circuit board can identify the “click” versus other sounds in the surgical space. The feedback system 100G can alternatively or additionally include the accelerometer 62G. The accelerometer 62G can be attached to the trocar 14G (or positioned within the trocar 14G), and the accelerometer 62G is configured to detect the vibrations caused when the anvil 12G is attached to the trocar 14G.

The accelerometer 62G is electrically wired back to the printed circuit board positioned with the housing 16. When the anvil 12G is attached to the trocar 14G, the vibrations from the retaining clip 42G snapping and contacting the trocar 14G are detected with the accelerometer 62G, which is in close proximity to the interface and the retaining clip 42G. The algorithms of the printed circuit board continuously monitor for the detection of the vibrations identified by the accelerometer 62G, and when the vibrations are above a predetermined threshold value, it is an indication that the anvil 12G have been properly attached to the trocar 14G. The printed circuit board then communicates and outputs a notification that the anvil 12G is attached to the trocar 14G. The notification to the user can be an audible notification and/or a light or message on the display 34, among other options not specifically listed. The feedback system 100G provides another feedback solution that communicates to the surgeon that the anvil 12G is properly attached to the trocar 14G before closing and firing of the circular stapler 10.

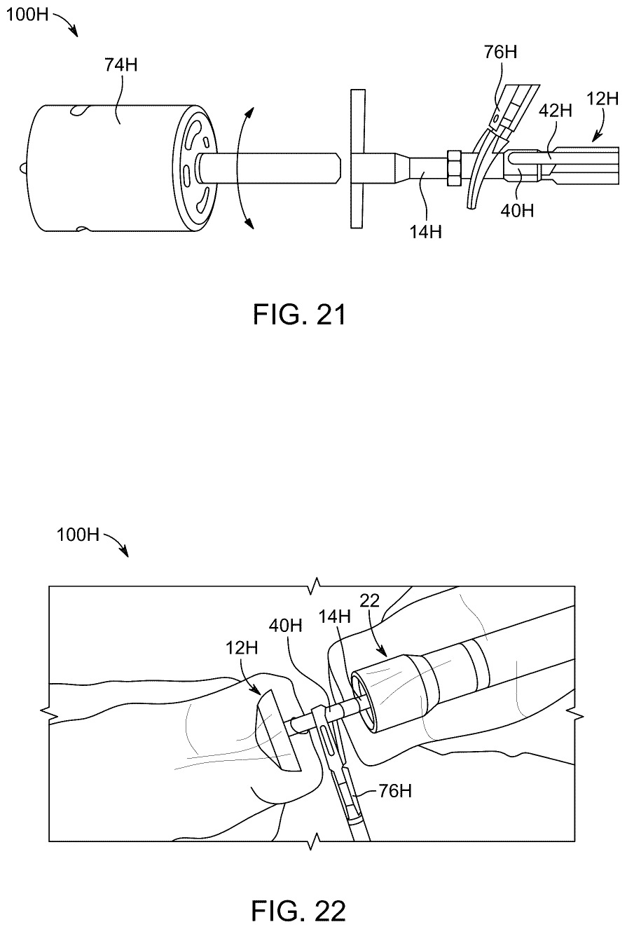

FIG. 21 is a side view of another embodiment of a feedback system 100H of the circular stapler 10 including an electronic verification system 72H and an actuator 74H. FIG. 22 is a side view of the feedback system 100H of FIG. 21 in use or operation. The feedback system 100H includes a motorized or actuated trocar 14H, such that the actuator 74H is activated to axially extend and retract the trocar 14H relative to the stapling head 22. The actuator 74H is communicatively coupled to the electronic verification system 72H, which electronic verification system 72H can be implemented within a printed circuit board (not shown) within the housing 16 of the circular stapler 10. The electronic verification system 72H is configured to monitor the torques experienced by the actuator 74H during extension and retraction of the trocar 14H. As illustrated in FIGS. 21-22, a user can use an anvil grasper 76H to grab the anvil shaft 40H of the anvil 12H. Then the user can guide the anvil shaft 40H onto the trocar 14H to attach the anvil 12H to the trocar 14H, with the retaining clip 42H snapping and latching onto the trocar 14H. Then a powered closure is initiated by the user, which activates the actuator 74H to begin axially translating the anvil 12H towards the stapling head 22. During the powered closure, the electronic verification system 72H monitors and determines if the torque versus position curve is representative of the expected resistance from the anvil 12H pulling on the proximal end of the colon or other tissue.

More specifically, the user can initiate a “check” function from an operating console to ensure the anvil 12H is properly attached to the trocar 14H, in which the actuator 74H completes a sequence of multiple small steps (i.e., small rotations in either direction). The electronic verification system 72H assess the monitored torque thresholds on the actuator 74H, and if thresholds exceed a predetermined threshold value (both pushing and pulling closure), this is an indication that the anvil 12H is attached to the trocar 14H. The printed circuit board then communicates and outputs a notification that the anvil 12H is attached to the trocar 14H. The notification can be an audible notification and/or a light or message on the display 34, among other options not specifically listed. The feedback system 100H provides another feedback solution that communicates that the anvil 12H is properly attached to the trocar 14H before closing and firing of the circular stapler 10. As such, detection of the connection of the anvil 12H to the trocar 14H can include comparing a torque response of the trocar 14H against a threshold torque value, which torque response is based on a predetermined sequence of motions of the trocar 14H.

The circular stapler 10 of the present disclosure alleviates the issues of determining whether the anvil 12 is properly connected to the trocar 14 by providing feedback to the surgeon that the anvil 12 is properly attached to the trocar 14 before closing and firing of the circular stapler 10. Several different embodiments of feedback systems are provided, with each feedback system providing an indication to the surgeon that the anvil 12 is properly attached to the trocar 14 before closing and firing of the circular stapler 10. As will be appreciated by those having skill in the art, the circular stapler 10 of the present disclosure provides many advantages over previously known circular staplers.

Having thus described the present embodiments in detail, it is to be appreciated and will be apparent to those skilled in the art that many physical changes, only a few of which are exemplified in the detailed description of the disclosure, could be made without altering the inventive concepts and principles embodied therein. It is also to be appreciated that numerous embodiments incorporating only part of the preferred embodiment are possible which do not alter, with respect to those parts, the inventive concepts and principles embodied therein.

The present embodiment and optional configurations are therefore to be considered in all respects as exemplary and/or illustrative and not restrictive, the scope of the disclosure being indicated by the appended claims rather than by the foregoing description, and all alternate embodiments and changes to this embodiment which come within the meaning and range of equivalency of said claims are therefore to be embraced therein.

What is claimed is:

1. A circular stapler comprising:
a housing with a handle extending from the housing;
a shaft coupled to and extending from the housing in a different direction than the handle;
a stapling head coupled to a distal end of the shaft, the stapling head being configured to cut tissue and dispense one or more staples into tissue;
an adjustment knob rotatably coupled to the housing, the adjustment knob being coupled to a first end of a pushrod extending through the housing and the shaft;
a trocar coupled to a second end of the pushrod, the trocar being positioned within and axially translatable relative to the stapling head;
an anvil removably coupled to a distal end of the trocar; and
a feedback system disposed within the stapling head, wherein the feedback system is configured to detect connection of the anvil to the trocar before retraction of the trocar into the stapling head, the feedback system being a lock that is pivotally connected to the trocar.

2. The circular stapler according to claim 1, wherein the lock is connected to an outer radial surface of the trocar.

3. The circular stapler according to claim 1, wherein the lock prevents retraction of the trocar into the stapling head when the anvil is disconnected from the trocar.

4. The circular stapler according to claim 1, wherein the lock is a spring biased lock that extends radially outwards from the trocar upon the trocar extending a specified distance out from the stapling head.

5. A circular stapler comprising:
a housing with a handle extending from the housing;
a shaft coupled to and extending from the housing in a different direction than the handle;
a stapling head coupled to a distal end of the shaft, the stapling head being configured to cut tissue and dispense one or more staples into tissue;
an adjustment knob rotatably coupled to the housing, the adjustment knob being coupled to a first end of a pushrod extending through the housing and the shaft;
a trocar coupled to a second end of the pushrod, the trocar being positioned within and axially translatable relative to the stapling head;
an anvil removably coupled to a distal end of the trocar; and
a feedback system disposed within the housing or the stapling head, wherein the feedback system comprises at least one sensor configured to detect connection of the anvil to the trocar.

6. The circular stapler according to claim 5, wherein detection of the connection of the anvil to the trocar comprises continuously monitoring one or more of audio signals, frequency signals or ranges, vibration signals, acceleration signals, friction signals, and torque signals.

7. The circular stapler according to claim 6, wherein the one or more audio signals, frequency signals or ranges, vibration signals, acceleration signals, friction signals, and torque signals being lower than a predetermined threshold value indicates connection of the anvil to the trocar.

8. The circular stapler according to claim 6, wherein the one or more audio signals, frequency signals or ranges, vibration signals, acceleration signals, friction signals, and torque signals being greater than a predetermined threshold value indicates nonconnection of the anvil to the trocar.

9. The circular stapler according to claim 5, wherein the detection of the anvil connection to the trocar comprises one or more mechanical features on the anvil or the trocar which generates a signature signal which is compared against expected signals to confirm anvil connection to the trocar.

10. The circular stapler according to claim 5, wherein the at least one sensor is a friction sensor.

11. The circular stapler according to claim 5, wherein the at least one sensor is an accelerometer.

12. The circular stapler according to claim 5, wherein the at least one sensor is a microphone.

13. The circular stapler according to claim 5, wherein the at least one sensor is a piezo-sensing element.

14. The circular stapler according to claim 5, wherein the at least one sensor is a torque sensor.

15. The circular stapler according to claim 5, wherein the at least one sensor is an electrical switch.

16. The circular stapler according to claim 15, wherein the trocar includes an undulating outer surface along an axial length of the trocar, and wherein the electrical switch contacts the undulating outer surface of the trocar.

17. A circular stapler comprising:
a housing with a handle extending from the housing;
a shaft coupled to and extending from the housing in a different direction than the handle;
a stapling head coupled to a distal end of the shaft, the stapling head being configured to cut tissue and dispense one or more staples into tissue;
an adjustment knob rotatably coupled to the housing, the adjustment knob being coupled to a first end of a pushrod extending through the housing and the shaft;
a trocar coupled to a second end of the pushrod, the trocar being positioned within and axially translatable relative to the stapling head;
an anvil removably coupled to a distal end of the trocar; and
a feedback system disposed within the housing or the stapling head, wherein the feedback system is configured to detect connection of the anvil to the trocar before retraction of the trocar into the stapling head, wherein detection of the connection of the anvil to the trocar comprises comparing a torque response of the trocar against a threshold torque value, which torque response is based on a predetermined sequence of motions of the trocar.

18. The circular stapler according to claim 17, wherein the torque response being lower than a predetermined threshold value indicates connection of the anvil to the trocar.

19. The circular stapler according to claim 17, wherein the torque response being greater than a predetermined threshold value indicates nonconnection of the anvil to the trocar.

20. The circular stapler according to claim 17, wherein the predetermined sequence of motions comprises a partial rotation of the trocar in at least one of a first direction and a second direction.
